(19) United States
(12) Patent Application Publication
(10) Pub. No.: US 2026/0306488 A1
(43) Pub. Date: Oct. 8, 2026

(54) FUNCTIONAL PROTECTIVE AGENT FOR ORGAN OR TISSUE

(71) Applicants: TOHOKU UNIVERSITY, Miyagi (JP); JELLICE Co., Ltd., Miyagi (JP)

(72) Inventors: Takaaki Abe, Miyagi (JP); Koichi Kikuchi, Miyagi (JP); Toru Nakayama, Miyagi (JP); Daiki Oikawa, Kyoto (JP); Noriaki Numata, Miyagi (JP); Shoko Yamamoto, Miyagi (JP); Yo Matsumoto, Miyagi (JP); Fumihiro Yambe, Miyagi (JP)

(21) Appl. No.: 19/484,943

(22) PCT Filed: May 24, 2024
(86) PCT No.: PCT/JP2024/019139

§ 371 Date: Nov. 17, 2025

(30) Foreign Application Priority Data
May 26, 2023 (JP) 2023-086860

(51) Int. Cl. A61K 31/4985; A61P 1/16; A61P 13/12

(52) U.S. Cl. CPC A61K 31/4985; A61P 1/16; A61P 13/12

(57) ABSTRACT

An object of the present invention is to provide a low-molecular-weight compound that is highly safe when ingested, and has an effect of preventing or improving dysfunction of an organ or a tissue. One or more compounds selected from a compound of the following formula (I), and a physiologically acceptable salt thereof are used as an agent for preventing or improving dysfunction of an organ or a tissue:

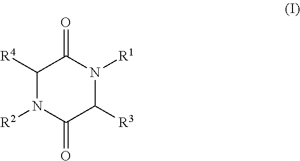

[wherein R¹ and R² each independently represent H or a C1-C6 alkyl group, R³ and R⁴ each independently represent H; or a C1-C6 alkyl group optionally having at least one group selected from the group consisting of a hydroxy group, a phenyl group, a hydroxyphenyl group, a S—CH₃ group, a NH₂ group, a carboxy group, a carbamoyl group, a guanidino group, and a N-containing heteroaryl group, R¹ and R³ optionally form a ring via a C2-C4 alkylene group optionally having a hydroxy group, and R² and R⁴ optionally form a ring via a C2-C4 alkylene group optionally having a hydroxy group.]

### TECHNICAL FIELD

The present invention relates to an agent for preventing or improving, etc. dysfunction of an organ or a tissue (hereinafter sometimes referred to as an “organ, etc.”) comprising a specific 2,5-diketopiperazine or a derivative thereof (hereinafter sometimes referred to as a “present diketopiperazine compound”) as an active ingredient.

### BACKGROUND ART

The kidney is a vital organ that maintains biological homeostasis by regulating the concentrations of component of bodily fluids through excretion and reabsorption, and also has functions as an endocrine organ producing and secreting physiologically active substances such as active vitamin D, erythropoietin, and renin. When the function of the kidney, which plays such a crucial role, is damaged and degraded, various pathological conditions are caused and a wide range of kidney diseases occur. These kidney diseases include nephropathies closely associated with diabetes, obesity, and abnormal lipid metabolism, and diabetic nephropathy, in particular, which originates from diabetes, is difficult to prevent progression thereof even with strict diabetic control after diagnosis, and frequently progresses to renal failure, and in addition, the number of patients suffering from this disease is not small.

2,5-Diketopiperazine (also referred to as a “cyclic dipeptide”) is a dipeptide with a cyclic structure formed by dehydration-condensation of an amino group and a carboxyl group present at the end of a straight-chain dipeptide, and in recent years, various physiological activities thereof have been drawing attention. For example, it has been reported that a specific diketopiperazine has a function of improving a moisturizing function of the skin (Patent Document 1), has a function of activating or inhibiting the activity of a sympathetic nerve in the skin (Patent Document 2), and has a vasodilatory effect (Patent Document 3). However, it has not been previously known that 2,5-diketopiperazine is effective against dysfunction of an organ or the like.

### PRIOR ART DOCUMENTS

### Patent Documents

Patent Document 1: International Publication No. WO 2016/063901
Patent Document 2: Japanese unexamined Patent Application Publication No. 2022-48337
Patent Document 3: Japanese unexamined Patent Application Publication No. 2018-16612

### SUMMARY OF THE INVENTION

### Object to be Solved by the Invention

An object of the present invention is to provide a low-molecular-weight compound that is highly safe when ingested, and has an effect of preventing or improving dysfunction of an organ, etc.

### Means to Solve the Object

For solving the aforementioned problem, the present inventors have continued to conduct diligent research. In the process, it has been found that a specific 2,5-diketopiperazine has an effect of improving dysfunction of an organ, etc. caused by a disease, etc. such as renal failure or diabetes, and as a result, the present invention has been accomplished.

Specifically, the present invention provides the following:

[1] An agent for preventing or improving dysfunction of an organ or a tissue, comprising one or more compounds selected from the group consisting of a compound of the following formula (I), and a physiologically acceptable salt thereof:

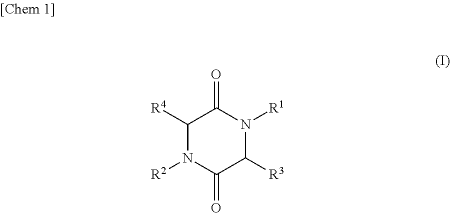

In formula (I), R¹ and R² each independently represent H or a C1-C6 alkyl group, R³ and R⁴ each independently represent H; or a C1-C6 alkyl group optionally having at least one group selected from the group consisting of a hydroxy group, a phenyl group, a hydroxyphenyl group, a S—CH₃ group, a NH₂ group, a carboxy group, a carbamoyl group, a guanidino group, and a N-containing heteroaryl group, R¹ and R³ optionally form a ring via a C2-C4 alkylene group optionally having a hydroxy group, and R² and R⁴ optionally form a ring via a C2-C4 alkylene group optionally having a hydroxy group.

[2] The agent according to [1] above, wherein the compound of formula (I) is a compound of the following formula (I-1)

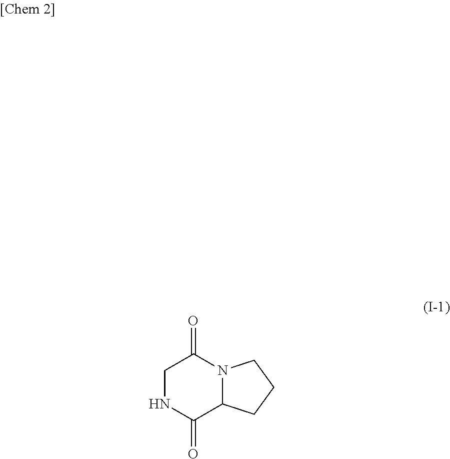 [SMILES: O=C1NCC(=O)N2CCCC12]

[3] The agent according to [1] or [2] above, wherein the organ is kidney or liver.

[4] The agent according to any one of [1] to [3] above, for use in preventing or improving dysfunction of an organ or a tissue in a mammal suffering from renal failure or diabetes.

[5] The agent according to any one of [1] to [4] above that is a pharmaceutical, a pet food, or a food/drink product.

Other embodiments of the present invention can include:
a method for preventing or improving (treating) dysfunction of an organ, etc., comprising a step of ingesting (administering) a present diketopiperazine compound to a mammal in need of prevention or improvement (treatment) of dysfunction of an organ, etc.;
a present diketopiperazine compound for use in prevention or improvement (treatment) of dysfunction of an organ, etc.; and
use of a present diketopiperazine compound for producing an agent for preventing or improving (treating) dysfunction of an organ, etc.

### Effect of the Invention

A present diketopiperazine compound has an effect of improving dysfunction of an organ, etc. caused by a disease such as renal failure or diabetes. Besides, the present diketopiperazine compound encompasses 2,5-diketopiperazines derived from food, and hence is highly safe when ingested. Therefore, when the present diketopiperazine compound is ingested, it is possible to protect the function of an organ, etc., prevent or treat a disease such as renal failure or diabetes, and reduce the risk of aggravation of the disease.

### BRIEF DESCRIPTION OF DRAWINGS

FIG. 1 is a diagram illustrating the results (mean±standard deviation) of measurement, performed in Example 1, of a concentration of a uremic substance (creatinine) in a “plasma samples after 2-week feeding” of three groups (a normal diet group [illustrated as “Control” in the diagram]), an adenine-fed control group [illustrated as “RF” in the diagram], and an adenine-fed cGP group [illustrated as “RF-c(GP)!” in the diagram]. In the drawing, “*” and “**” respectively indicate statistically significant differences as determined by Dunnett's test (p<0.05 and p<0.01).

FIG. 2 is a diagram illustrating the results of an analysis, performed in Example 1, of kidney tissues of the three groups described above after 2-week feeding. FIG. 2A illustrates the results (mean±standard deviation) of the analysis of a ratio of renal tubules in the renal cortex (%). In the drawing, “**” indicates a statistically significant difference as determined by Dunnett's test (p<0.01). FIG. 2B illustrates representative examples of a histological image of the kidney tissues used for the analysis of FIG. 2A. A darker portion indicates normal renal parenchyma, and a lighter portion indicate a fibrotic region.

FIG. 3 is a diagram illustrating the results (mean±standard deviation) of measurement, performed in Example 1, of an ALT (alanine aminotransferase) concentration (FIG. 3A) and a total cholesterol concentration (FIG. 3B) in “plasma samples after 2-week feeding” of three groups (a normal diet group [illustrated as “Ctrl” in the diagram], an adenine-fed control group [illustrated as “RF” in the diagram], and an adenine-fed cGP group [illustrated as “RF-c(GP)” in the diagram]). In the drawing, “*” indicates a statistically significant difference as determined by Dunnett's test (p-value<0.05).

FIG. 4 is a diagram illustrating the results of an analysis, performed in Example 2, by a hematoxylin and eosin (HE) staining method, of liver tissues of two groups (a diabetes model normal diet group [illustrated as “dbdb” in the drawing] and a diabetes model cGP group [illustrated as “dbdb-c(GP)” in the drawing]) after 6-week feeding. White spotty portions seen within the tissues are fat droplets.

FIG. 5 is a diagram illustrating the results of measurement, performed in Example 2, of a creatinine concentration in “plasma samples after 6-week feeding” of four groups (a wild-type normal diet group [illustrated as “Normal” in the drawing], a wild-type cGP group [illustrated as “Normal-c(GP)” in the drawing], a diabetes model normal diet group [illustrated as “dbdb” in the drawing], and a diabetes model cGP group [illustrated as “dbdb-c(GP)” in the drawing]). In the drawing, “*”, “**” and “***” respectively indicate statistically significant differences as determined by Dunnett's test (p<0.05, p<0.01, and p<0.001).

FIG. 6 is a diagram illustrating the results of measurement, performed in Example 2, of concentrations of three substances (creatinine [FIG. 6A], phenyl sulfate [FIG. 6B], and Ac6Lys [FIG. 6C]) in “plasma samples after 0-week feeding” (illustrated as “0 w” in the drawing), and “plasma samples after 6-week feeding” (illustrated as “6 w” in the drawing) of the diabetes model cGP group. In the drawing, “*” indicates a statistically significant difference as determined by Paired t-test (p<0.05).

FIG. 7 is a diagram illustrating the results of measurement, performed in Example 3, of concentrations of two uremic substances (1-methyladenosine [m1A][FIG. 7A] and trimethylamine-N-oxide [TMAO] [FIG. 7B]) in plasma samples of two groups (a diabetes model normal diet group [illustrated as “dbdb” in the drawing] and a diabetes model cGP group [illustrated as “dbdb-c(GP)” in the drawing]) after 5-week feeding. In the drawing, “**” indicates a statistically significant difference as determined by Unpaired t-test (p<0.01).

### MODE OF CARRYING OUT THE INVENTION

An agent for preventing or improving of the present invention is an agent that comprises one or more compounds selected from a present diketopiperazine compound, namely, a compound of the following formula (I), and a physiologically acceptable salt thereof, and is specified for use of “preventing or improving dysfunction of an organ or a tissue” (hereinafter, sometimes referred to as the “present preventive agent/improving agent”).

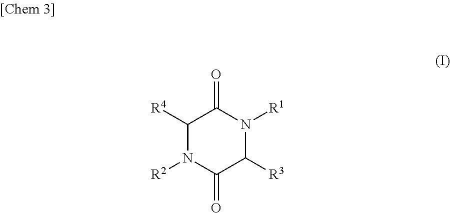

In formula (I),
R¹ and R² each independently represent H or a C1-C6 alkyl group,
R³ and R⁴ each independently represent H; or a C1-C6 alkyl group optionally having at least one group selected from the group consisting of a hydroxy group, a phenyl group, a hydroxyphenyl group, a S—CH₃ group, a NH₂ group, a carboxy group, a carbamoyl group, a guanidino group, and a N-containing heteroaryl group (namely, a C1-C6 alkyl group; or a C1-C6 alkyl group having at least one group selected from the group consisting of a hydroxy group, a phenyl group, a hydroxyphenyl group, a S—CH₃ group, a NH₂ group, a carboxy group, a carbamoyl group, a guanidino group, and a N-containing heteroaryl group),
R¹ and R³ optionally form a ring via a C2-C4 alkylene group optionally having a hydroxy group, and
R² and R⁴ optionally form a ring via a C2-C4 alkylene group optionally having a hydroxy group.

In formula (I), examples of the C1-C6 alkyl group used as R¹ to R⁴, and the C1-C6 alkyl group used as R² can include a methyl group, an ethyl group, a n-propyl group, an i-propyl group, a n-butyl group, a s-butyl group, an i-butyl group, a t-butyl group, a n-pentyl group, and a n-hexyl group.

In formula (I), the N-containing heteroaryl group, which is one of substituents of the C1-C6 alkyl group used as R³ and R⁴, encompasses a 5- to 7-membered monocyclic or polycyclic aromatic heterocycle having 1 to 4 nitrogen atoms as heteroatoms, and a fused ring in which a benzene ring is fused with a 5- to 7-membered heterocycle having 1 to 4 nitrogen atoms as heteroatoms. Examples thereof can include pyridinyl, imidazolyl, pyrimidinyl, pyrazolyl, triazolyl, pyrazinyl, tetrazolyl, pyrrolyl, quinolinyl, indolyl, benzimidazolyl, pyridazinyl, and triazinyl.

The present diketopiperazine compound encompasses an isomer such as a stereoisomer (such as an enantiomer) or a tautomer, a solvate, a hydrate, etc., and a mixture thereof.

Examples of the physiologically acceptable salt of the compound of formula (I) described above can include: an inorganic salt such as sulfate, pyrosulfate, bisulfate, sulfite, bisulfite, nitrate, phosphate, monohydrogen phosphate, dihydrogen phosphate, metaphosphate, pyrophosphate, chloride, bromide, iodide, or fluoride; an organic salt such as acetate, propionate, decanoate, caprylate, acrylate, formate, isobutyrate, caprate, heptanoate, propiolate, oxalate, malonate, succinate, suberate, fumarate, maleate, benzoate, chlorobenzoate, methylbenzoate, dinitrobenzoate, hydroxybenzoate, methoxybenzoate, phthalate, terephthalate, benzenesulfonate, toluenesulfonate, chlorobenzenesulfonate, xylenesulfonate, phenylacetate, phenylpropionate, phenylbutyrate, citrate, lactate, hydroxybutyrate, glycolate, malate, or tartrate; an alkali metal salt such as sodium salt and potassium salt; an alkaline earth metal salt such as calcium salt or magnesium salt; and ammonium salt.

Specific examples of the present diketopiperazine compound can include a compound of the following formula (I-1) (cGP [cyclo-glycyl-proline]); a compound of formula (I-2) (cGS [cyclo-glycyl-serine]); a compound of formula (I-3) (cGL [cyclo-glycyl-leucine]); a compound of formula (I-4) (cGE [cyclo-glycyl-glutamic acid]); a compound of formula (I-5) (cGA [cyclo-glycyl-alanine]); or a compound of formula (I-6) (cGF [cyclo-glycyl-phenylalanine]), and among these, the compound of the following formula (I-1) is preferred because the effect thereof has been verified in Examples described below.

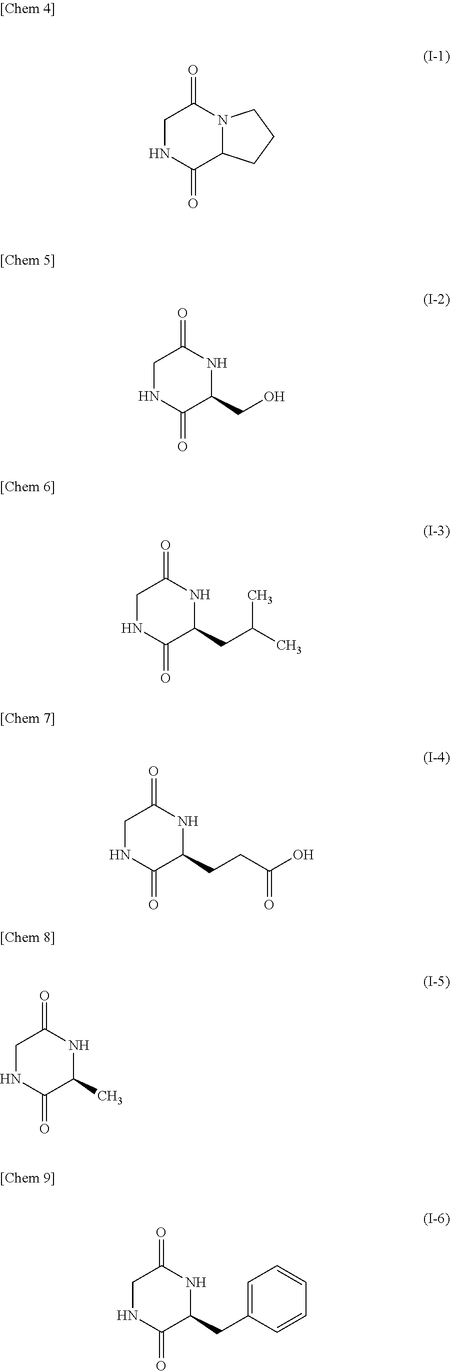

As the present diketopiperazine compound, a commercially available product may be used, or a product synthesized by a known method may be used.

A method for synthesizing the present diketopiperazine compound by a known method can be, for example, a method in which a dipeptide of the following formula (II) is heated in an organic solvent with water removed by distillation according to a method described in Japanese unexamined Patent Application Publication (Translation of PCT Application) No. 2003-531197.

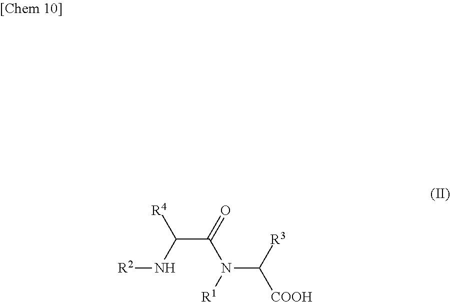

In formula (II), R¹ to R⁴ each have the same meaning as R¹ to R⁴ in formula (I).

The above organic solvent may be any solvent that forms a low-boiling azeotrope with water, and examples thereof can include acetonitrile, allyl alcohol, benzene, benzyl alcohol, n-butanol, 2-butanol, t-butanol, butyl acetate, carbon tetrachloride, chlorobenzene, chloroform, cyclohexane, 1,2-dichloroethane, diethyl acetal, dimethyl acetal, ethyl acetate, heptane, methyl isobutyl ketone, 3-pentanol, toluene, and xylene.

A temperature during the reaction is usually 50 to 200° C., and preferably 80 to 150° C. The pH at which cyclization occurs is usually 2 to 9, and preferably 3 to 7.

A dipeptide of formula (II) can be obtained, for example, by a dehydration condensation reaction between glycine and another amino acid or a derivative thereof.

Alternatively, the dipeptide can be produced by heating an aqueous solution containing a straight-chain dipeptide or a straight-chain tripeptide under a pressure of 0.5 MPa or less at 70 to 100° C. according to a method described in Japanese Patent No. 5456876.

2,5-Diketopiperazine is known to be contained in fruits and processed foods involving fermentation or heating processes, such as a blackcurrant, cordyceps, cheese, beef stew, and chicken essence, and can be obtained as a part of an extract or concentrate from such a food.

Herein, examples of a mammal can include a human and a non-human mammal (for example, a monkey; a mouse; a rat; a pet such as a dog or a cat; and livestock such as a rabbit, a pig, a horse, a cow, sheep, a goat, or deer).

When the present preventive agent/improving agent is ingested by a non-human mammal or administered to a non-human mammal, the present diketopiperazine compound may be used singly as a feed for the non-human mammal, or, furthermore, may be mixed with an additive to be used in the form of a composition (i.e., a feed composition for the non-human mammal). Furthermore, when the present preventive agent/improving agent is ingested by a non-human mammal or a human, or administered to a non-human mammal or a human, the present diketopiperazine compound may be used singly as a food/drink product, or a pharmaceutical (preparation), or, furthermore, may be mixed with an additive to be used in the form of a composition (a food/drink composition or a pharmaceutical composition). Examples of the above food/drink product include health food (such as a functional food, a nutritional supplement, a health supplement, a fortified food, a nutrient adjusted food, and a supplement), health promoting food (such as a food for specified health uses, a food with nutrient function claims, and a food with functional claims), and herbal medicines. The present preventive agent/improving agent is preferably a pharmaceutical, a pet food, or a food/drink product.

Herein, examples of the “organ or tissue” can include the large intestine (colon or rectum), stomach, liver, heart, brain, spinal cord, lung, esophagus, duodenum, small intestine, skin, prostate, bladder, uterus, kidney, pancreas, spleen, trachea, bronchi, gallbladder, bile duct, bone, gum, adrenal gland, thyroid gland, and blood vessel, and suitable examples can include kidney and liver because the effect thereof has been verified in Examples described below.

Herein, the “dysfunction of an organ or a tissue” means a state where the function of an organ or a tissue is lower than normal due to a disease, etc., such as a state in which abnormal test values (for example, blood concentrations of uremic substances [for example, creatinine, phenyl sulfate, 1-methyladenosine, trimethylamine-N-oxide [TMAO], indoxyl sulfate, and blood urea nitrogen [BUN]], a blood concentration of a factor leading to renal failure in a patient with type 1 diabetes [for example, Ac6Lys], an estimated glomerular filtration rate [eGFR], a urinary protein concentration, a blood concentration of a liver function marker [for example, AST [aspartate aminotransferase], ALT, ALP [alkaline phosphatase], γGTP, etc.], or a blood concentration of lipid [for example, triglycerides [TG], or total cholesterol]) are shown in health checkup (which state is, for example, a state showing an increase in the blood concentration of a uremic substance, an increase in the blood concentration of a factor leading to renal failure in a patient with type 1 diabetes, a decrease in eGFR, an increase in the urinary protein concentration [positive for proteinuria], an increase in the blood concentration of a liver function marker, or an increase in the blood concentration of lipid). Examples of the above disease can include: digestive system abnormality such as stomatitis and enteritis; nervous system abnormality such as multiple neuritis and (developmental) coordination disorder; circulatory system abnormality such as arteriosclerosis, anemia, erythropoiesis disorder, hypertension, ischemic heart disease, pericarditis, myocarditis, (blood) coagulation abnormality, heart failure, and cardiovascular disorder; skin abnormality (disease) such as pigmentation, pruritus, psoriasis, atopic dermatitis, and hair loss; kidney dysfunction such as albuminuria, renal failure (acute renal failure or chronic renal failure), acute tubular necrosis, renal anemia, tubulointerstitial disorder, acute nephritis, chronic nephritis, nephrosis, diabetic nephropathy, arteriosclerotic nephropathy, renal bone abnormality (for example, renal osteodystrophy), and fluid retention; dyslipidemia (hyperlipidemia); diabetes; stroke; myocardial infarction; cancer; autism; immunodeficiency; bone abnormality; hyperparathyroidism; insulin resistance; malnutrition; inflammation (for example, chronic inflammation); cellular damage; and tremors. The above disease also includes symptom or disease in which LPS (Lipopolysaccharide) is involved, such as IgA nephropathy, polycystic kidney disease, liver dysfunction (for example, fulminant hepatitis, fatty liver, NASH, NAFLD), and cancer (for example, biliary tract carcinogenesis); inflammatory bowel disease such as ulcerative colitis and Crohn's disease; arteriosclerosis-related disease; autoimmune disease such as lupus, scleroderma, and rheumatism; autism; Parkinson's disease; Alzheimer's disease; and sarcopenia. Herein, the “uremic substance” means a substance that is excreted by a healthy kidney (such as a waste product or toxin), and increases (accumulates) in the blood when the excretory function is degraded due to some cause such as kidney dysfunction.

An ingestion (administration) subject of the present preventive agent/improving agent is not particularly limited as long as it is a mammal, and is usually a mammal in need of prevention or improvement (treatment) of dysfunction of an organ, etc., and more specifically, is a mammal in a state where the function of an organ or a tissue has been degraded due to the diseases mentioned above, etc., and a suitable example can include a mammal suffering from renal failure or diabetes because the effect thereof has been verified in Examples described below. When the present diketopiperazine compound contained in the present preventive agent/improving agent is ingested, test values in health checkup are improved, and the function of the organ or the tissue is protected, and the compound contributes to the prevention or treatment of the above diseases, as well as the reduction of the risk of aggravation of the above diseases. Therefore, the present preventive agent/improving agent can be used to improve test values in health checkup; protect the function of an organ or a tissue; prevent or treat the above diseases; or reduce the risk of aggravation of the above diseases.

The dosage form of the present preventive agent/improving agent can be oral administration for ingesting (administration) in the form of a powder, a granule, a tablet, a capsule, a syrup, a jelly, a gum, a troche, a gummy, a film, etc., or parenteral administration for ingesting (administration) in the form of a topical skin application, a transdermal patch, a transmucosal agent, a nasal spray, an enteral preparation, an injection, a suppository, an inhalant, a patch, etc. The parenteral administration includes administration, in the form of a liquid, a semi-solid preparation, etc., by tube feeding via a stoma, such as a gastrostomy, or tube feeding using a nasogastric tube.

A present preventive agent/improving agent for oral ingestion (administration) can be a sustained-release preparation or an enteric-coated preparation. Such an enteric-coated preparation can be obtained, for example, by filling an enteric film (i.e., a film containing, as a main component, a base resistant to gastric acid and soluble in the small intestine [an enteric component]) with granules containing the active ingredient of the present diketopiperazine compound, or by coating, with an enteric film, a tablet obtained by adding a lubricant to granules containing the active ingredient of the present diketopiperazine compound, and tableting the resultant.

As a method for obtaining a present preventive agent/improving agent in a tablet form, for example, a method in which the active ingredient of the present diketopiperazine compound is mixed with any optional component (additive) blended if necessary, and the resultant mixture is compression-molded to obtain the tablet, or a method in which a tablet resulting from the compression molding is coated with an enteric component (method for obtaining the agent in the form of an enteric-coated agent) can be employed, and the latter method is preferred.

Examples of the above enteric component can include shellac, zein, hydroxymethylcellulose phthalate, carboxymethylcellulose, carboxymethylethylcellulose, cellulose acetate phthalate, a methacrylic acid copolymer, water-insoluble ethylcellulose, an aminoalkyl methacrylate copolymer, a brewer's yeast cell wall (for example, one available under product name Eastrap), tapioca starch, gelatin, and pectin, among which shellac is preferred. It can be confirmed whether or not an agent is an enteric-coated preparation by the disintegration test described in the Japanese Pharmacopoeia Fourteenth Edition.

An intake of the present diketopiperazine compound contained in the present preventive agent/improving agent is appropriately determined based on the age, weight, sex, symptoms, drug sensitivity, species, etc. of a mammal that is the ingestion (administration) subject, and for example, is a dosage within the range of 0.1 μg to 200 mg/kg (weight)/day in terms of a concentration of the present diketopiperazine compound. The present preventive agent/improving agent may be ingested once or a plurality of times (for example, 2 to 4 times) per day.

Herein, examples of the “additive” include blended ingredients such as a physiologically acceptable normal carrier, a binder, a stabilizer, an excipient, a diluent, a pH buffer, a disintegrant, an isotonic agent, a coating agent, a solubilizing agent, a lubricating agent, a glidant, a dissolution aid, a lubricant, a flavoring agent, a sweetener, a solvent, a gelling agent, a nutrient, various oils, a surfactant, a preservative, an antioxidant, a dispersing agent, a chelating agent, a thickener, a UV absorber, an emulsion stabilizer, a pH adjuster, a pigment, and a fragrance. Specific examples of such blended ingredients include water, saline, an animal fat and oil, a vegetable oil, lactose, starch, gelatin, crystalline cellulose, a gum, talc, magnesium stearate, hydroxypropylcellulose, polyalkylene glycol, polyvinyl alcohol, glycerin, vitamin, mineral, lactic acid bacteria, protein, and collagen.

The present preventive agent/improving agent may also contain another ingredient, in addition to the present diketopiperazine compound, as an ingredient for preventing or improving dysfunction of an organ, etc., but may not contain, in addition to the present diketopiperazine compound, any of the aforementioned ingredients (for example, a compound such as protein, an amino acid, DNA, RNA, or a polymer; or plant-derived extracts [for example, sesame lignan compounds]) because the present diketopiperazine compound singly exhibits an excellent inhibitory effect.

Hereinafter, the present invention will now be described more specifically by way of Examples, but the technical scope of the present invention is not limited to these examples.

### EXAMPLES

### Example 1

1. Confirmation that Present Compound Protects Kidney Function and Liver Function in Renal Failure Model

In order to confirm that a present compound has an effect of protecting kidney function and liver function in a renal failure model, 2,5-diketopiperazine (cGP; see Table 1) encompassed in the present compound was used for performing analysis in which the cGP is administered to a model mouse with chronic renal failure induced by feeding adenine (hereinafter referred to as the “adenine-fed renal failure model mouse”) to analyze the blood concentration of a uremic substance (creatinine), that is, an indicator of kidney function, and a ratio of renal tubules, as well as to analyze blood concentrations of three liver function markers (ALT, AST, and γGTP) and two lipids (total cholesterol and TG). The adenine-fed renal failure model mouse is a mouse that develops kidney damage because fed adenine crystallizes as insoluble 2,8-dihydroxyadenine in the renal tubules (Cozzolino, M. et al. Kidney Int. 64, 441-450 (2003), Tamagaki, K. et al. Nephrol. Dial. Transplant 21, 651-659 (2006)).

### 1-1. Method

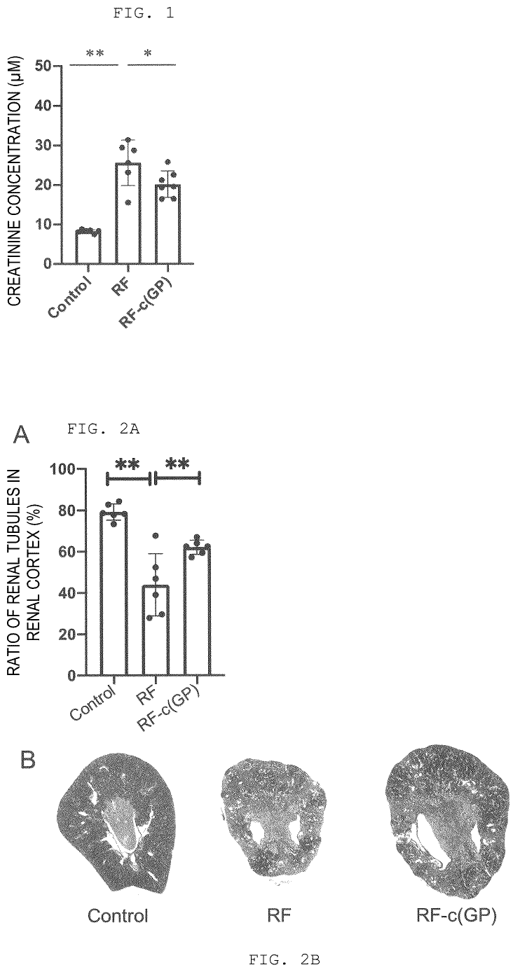

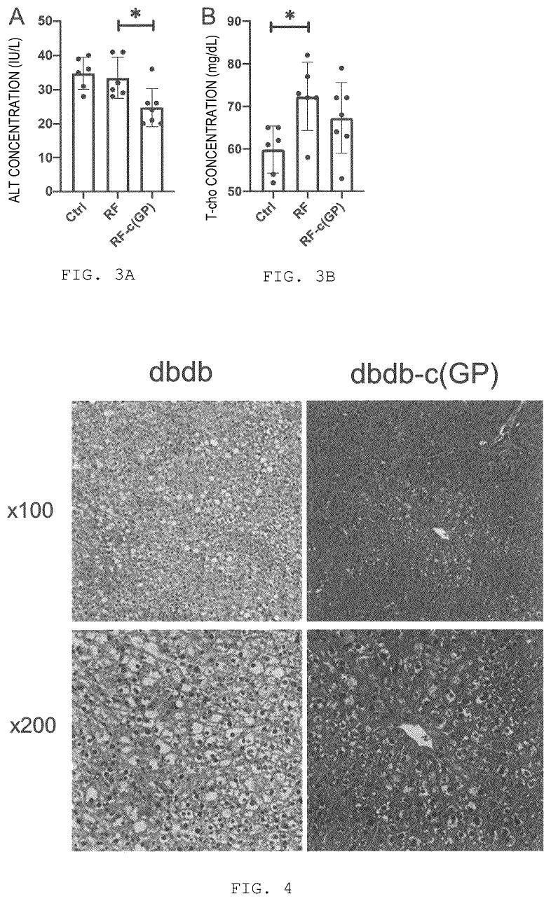

The analysis using an adenine-fed renal failure model mouse was performed according to the following procedures [1] through [5]:

[1] 8-Week old male C57BL/6 mice (purchased from Japan CLEA) were grouped into a normal diet group (n=6) to be fed a normal diet (CE-2, manufactured by Japan CLEA), and an adenine-fed group (n=12) to be fed the normal diet containing 0.2% adenine (manufactured by Wako).

[2] After feeding the mice for 6 weeks, the adenine-fed group was grouped into an adenine-fed control group (n=6) to be fed the normal diet, and an adenine-fed cGP group (n=6) to be fed the normal diet containing cGP. The normal diet containing cGP was prepared by mixing cGP (manufactured by Bachem) with the normal diet to a final concentration of 0.0025%.

[3] After feeding the mice for 2 weeks, blood samples were harvested from each of the three groups (the normal diet group, the adenine-fed control group, and the adenine-fed cGP group) to prepare plasma samples (referred to as “plasma samples after 2-week feeding”) according to a usual method.

[4] After feeding the mice for another week (3 weeks in total), blood samples were harvested from the above three groups, plasma samples (referred to as the “plasma samples after 3-week feeding”) were prepared according to a usual method, and then, kidney tissues were harvested, fixed in 10% neutral-buffered formalin, and embedded in paraffin, and the resultant paraffin-embedded kidney tissues were sliced to prepare kidney tissue sections.

[5] The thus obtained “plasma samples after 2-week feeding” were used to measure a blood concentration of a uremic substance (creatinine), that is, an indicator of kidney function, by mass spectrometry using a liquid chromatograph-tandem mass spectrometer (LC-MS/MS) (manufactured by Shimadzu Corporation) (see FIG. 1), and 10 μL of each of the “plasma samples after 2-week feeding” and the “plasma samples after 3-week feeding” was used to measure blood concentrations of three liver function markers (ALT [see FIG. 3A], AST, and γGTP) and two lipids (total cholesterol [see FIG. 3B] and TG) with DRI-CHEM 7000V (manufactured by Fujifilm Corporation). Besides, the kidney tissue sections prepared as described above were used to perform analysis by Masson's Trichrome Staining (MTS) method according to a usual method (see FIG. 2).

### 1-2 Results

It was revealed that the creatinine concentration in the “plasma samples after 2-week feeding” of the adenine-fed control group is significantly increased compared to the creatinine concentration in the “plasma samples after 2-week feeding” of the normal diet group (see FIG. 1). Furthermore, it was revealed that the ratio of renal tubules in the adenine-fed control group is significantly decreased compared to the ratio of renal tubules in the normal diet group (see FIG. 2A). These results show that in the adenine-fed control group (namely, the adenine-fed renal failure model mice), the reduction in kidney function due to renal failure led to an increase in blood concentration of the uremic substance (creatinine).

On the other hand, it was revealed that the creatinine concentration in the “plasma samples after 2-week feeding” of the adenine-fed cGP group is significantly decreased compared to the creatinine concentration in the “plasma samples after 2-week feeding” of the adenine-fed control group (see FIG. 1) Furthermore, it was revealed that the ratio of renal tubules in the adenine-fed cGP group is significantly increased compared to the ratio of renal tubules in the adenine-fed control group (see FIG. 2A). These results show that when a present compound group (cGP) is administered to an adenine-fed renal failure model mouse, the reduced kidney function is improved, resulting in a decrease in the elevated blood concentration of the uremic substance (creatinine).

Besides, it was revealed that the ALT concentration in the “plasma samples after 2-week feeding” of the adenine-fed cGP group is significantly decreased compared to the ALT concentration in the “plasma samples after 2-week feeding” of the adenine-fed control group (see FIG. 3A). Furthermore, it was revealed that the total cholesterol concentration in the “plasma samples after 2-week feeding” of the adenine-fed cGP group is significantly decreased compared to the total cholesterol concentration in the “plasma samples after 2-week feeding” of the adenine-fed control group (see FIG. 3B) These results show that when the present compound group (cGP) is administered to an adenine-fed renal failure model mouse, the liver function is improved as well as dyslipidemia is improved.

TABLE 1

| 2,5-Diketopiperazine | Chemical Structural Formula |
|---|---|
| cGP | 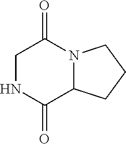 [SMILES: O=C1NCC(=O)N2CCCC12] |
| | (1-1) |

### Example 2

2. Confirmation that Present Compound Protects Kidney Function and Liver Function in Diabetes Model

Next, in order to confirm that the present compound protects kidney function and liver function in a diabetes model, cGP was administered to a diabetes model mouse (db/db mouse) to analyze blood concentrations of two uremic substances (creatinine and phenyl sulfate), as well as a blood concentration of a factor leading to renal failure in a patient with type 1 diabetes (N6-acetyllysine [Ac6Lys]), and additionally, pathological tissues of liver and blood concentrations of three liver function markers (ALT, AST, and γGTP) and two lipids (total cholesterol and TG) were analyzed.

### 2-1 Method

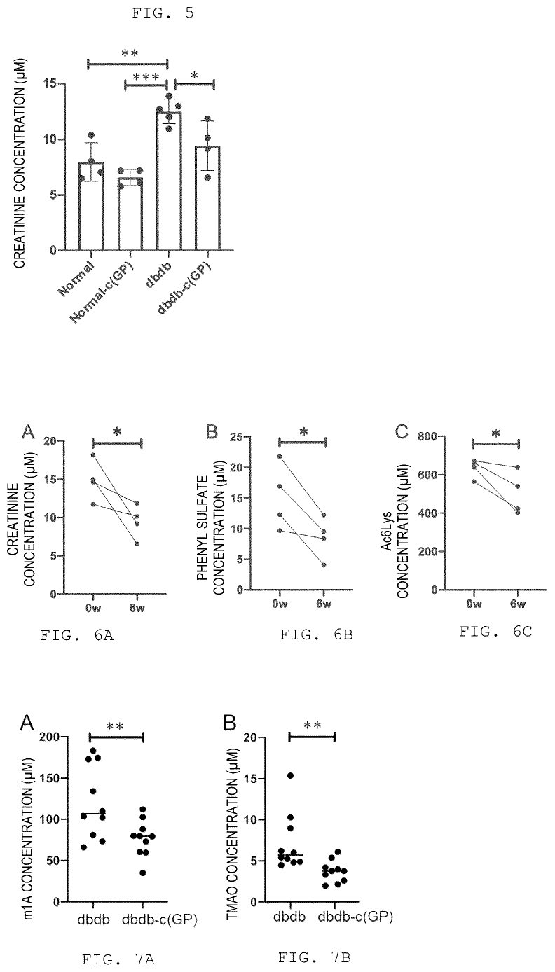

The analysis using a diabetes model mouse was performed according to the following procedures [1] through [3]:

[1] Wild-type mice (BKS-m+/m+ mice, purchased from Japan CLEA) having been fed a normal diet (CE-2, manufactured by Japan CLEA) were grouped into a wild-type normal diet group (n=5) to be fed the normal diet, and a wild-type cGP group (n=4) to be fed the normal diet containing cGP. Besides, diabetes model mice (BKS-db/db mice; purchased from Japan CLEAR) having been fed the normal diet were grouped into a diabetes model normal diet group (n=4) to be fed the normal diet, and a diabetes model cGP group (n=4) to be fed the normal diet containing cGP. Blood samples were harvested from the diabetes model cGP group, and plasma samples (referred to as the “plasma samples after 0-week feeding”) were prepared according to a usual method.

[2] After feeding the mice for 6 weeks, blood samples were harvested from the above four groups (the wild-type normal diet group, the wild-type cGP group, the diabetes model normal diet group, and the diabetes model cGP group), plasma samples (referred to as the “plasma samples after 6-week feeding”) were prepared according to a usual method, and then, liver tissues were harvested, fixed in 10% neutral-buffered formalin, and embedded in paraffin, and the resultant paraffin-embedded liver tissues were sliced to prepare liver tissue sections.

[3] The thus obtained “plasma samples after 0-week feeding” and “plasma samples after 6-week feeding” were used to measure blood concentrations of the three substances (creatinine, phenyl sulfate, and Ac6Lys) (see FIGS. 5 and 6), and blood concentrations of three liver function markers (ALT, AST, and γGTP) and two lipids (total cholesterol and TG) by mass spectrometry using LC-MS/MS (manufactured by Shimadzu Corporation). Besides, the liver tissue sections prepared as described above were used to perform analysis by Hematoxylin and Eosin (HE) staining method according to a usual method (see FIG. 4).

### 2-2 Results

It was revealed that fat droplets in the liver of the diabetes model cGP group are reduced compared to the fat droplets in the liver of the diabetes model normal diet group (see FIG. 4). This result shows that when the present compound group (cGP) is administered to a diabetes model mouse, fatty liver is improved.

It was revealed that the creatinine concentration in the “plasma samples after 6-week feeding” of the diabetes model normal diet group is significantly increased compared to the creatinine concentration in the “plasma samples after 6-week feeding” of the wild-type normal diet group and the wild-type cGP group (see FIG. 5). This result shows that in the diabetes model normal diet group, reduction in kidney function due to diabetes led to an increase in blood concentration of the uremic substance (creatinine).

On the other hand, it was revealed that the creatinine concentration in the “plasma samples after 6-week feeding” of the diabetes model cGP group is significantly decreased compared to the creatinine concentration in the “plasma samples after 6-week feeding” of the diabetes model normal diet group (see FIG. 5). This result shows that when the present compound group (cGP) is administered to a diabetes model mouse, the reduced kidney function is improved, resulting in a decrease in the elevated blood concentration of the uremic substance (creatinine).

Furthermore, as a result of measurement of blood concentrations of three substances (creatinine, phenyl sulfate, and Ac6Lys) using the “plasma samples after 0-week feeding” and the “plasma samples after 6-week feeding” of the diabetes model cGP group, it was revealed that the blood concentrations of all of these substances are significantly lower at week 6 after feeding compared to week 0 after feeding (see FIG. 6). This result shows that when the present compound group (cGP) is administered to a diabetes model mouse, the reduced kidney function is improved and the risk of renal failure is reduced, and as a result, the blood concentrations of the uremic substances (creatinine and phenyl sulfate) decrease, as well as the blood concentration of the factor leading to renal failure in a patient with type 1 diabetes (Ac6Lys) decrease.

### Example 3

3. Confirmation that Present Compound Reduces Chronic Inflammation and Resulting Cellular Damage, and Prevents Vascular Complications in Diabetes Model

Next, in order to confirm that the present compound reduces chronic inflammation and resulting cellular damage, and prevents vascular complication in a diabetes model, cGP was administered to a diabetes model mouse (db/db mouse) to analyze blood concentrations of two uremic substances (1-methyladenosine [m1A] and trimethylamine-N-oxide [TMAO]).

### 3-1 Method

The analysis using a diabetes model mouse was performed according to the following procedures [1] through [3]:

[1] Diabetes model mice (BKS-db/db mice, purchased from Japan CLEA) having been fed a normal diet (CE-2, manufactured by Japan CLEA) were grouped into a diabetes model normal diet group (n=10) to be fed the normal diet, and a diabetes model cGP group (n=10) to be fed the normal diet containing cGP.

[2] After feeding the mice for 5 weeks, blood samples were harvested from the above two groups (the diabetes model normal diet group, and the diabetes model cGP group), and plasma samples were prepared according to a usual method.

[3] The thus prepared plasma samples were used to measure blood concentrations of two uremic substances (m1A and TMAO) by mass spectrometry using LC-MS/MS (manufactured by Shimadzu Corporation) (see FIG. 7).

### 3-2 Results

It was revealed that the m1A concentration in the plasma samples of the diabetes model cGP group are significantly reduced compared to the m1A concentration in the plasma samples of the diabetes model normal diet group (see FIG. 7A). Since m1A is known to be an early-stage marker that increases when cell damage occurs, it shows that when the present compound group (cGP) is administered to a diabetes model mouse, chronic inflammation and the resulting cellular damage are reduced.

Besides, it was revealed that the TMAO concentration in the plasma samples of the diabetes model cGP group is significantly reduced compared to the TMAO concentration in the plasma samples of the diabetes model normal diet group (see FIG. 7B). Since TMAO is known to be a promoter of atherosclerosis and has been reported to be correlated to cardiovascular events and life expectancy, it shows that when the present compound group (cGP) is administered to a diabetes model mouse, vascular complications, which are one of the main complications of diabetes, can be prevented.

### INDUSTRIAL APPLICABILITY

The present invention is useful for prevention and treatment of a disease such as renal failure or diabetes, as well as for reducing the risk of aggravation of such a disease.

1. A method for preventing or improving dysfunction of an organ or a tissue, comprising making a mammal in need of prevention or improvement of dysfunction of an organ or a tissue ingest one or more compounds selected from the group consisting of a compound of the following formula (I), and a physiologically acceptable salt thereof: 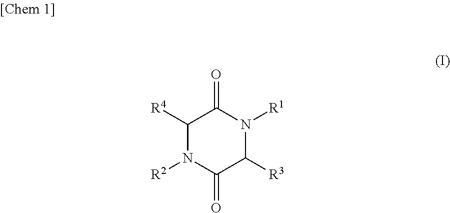
[wherein
R¹ and R² each independently represent H or a C1-C6 alkyl group,
R³ and R⁴ each independently represent H; or a C1-C6 alkyl group optionally having at least one group selected from the group consisting of a hydroxy group, a phenyl group, a hydroxyphenyl group, a S—CH₃ group, a NH₂ group, a carboxy group, a carbamoyl group, a guanidino group, and a N-containing heteroaryl group,
R¹ and R³ optionally form a ring via a C2-C4 alkylene group optionally having a hydroxy group, and
R² and R⁴ optionally form a ring via a C2-C4 alkylene group optionally having a hydroxy group.]

2. The method according to claim 1, wherein the compound of formula (I) is a compound of the following formula (I-1):

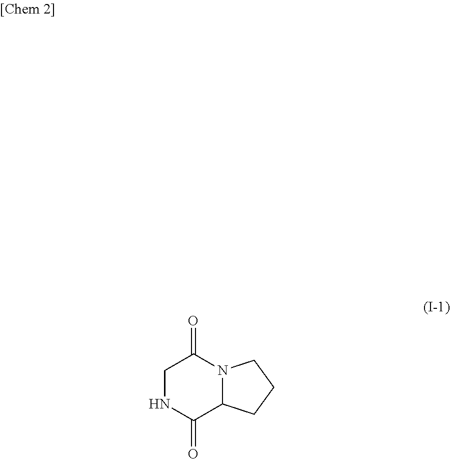 [SMILES: O=C1NCC(=O)N2CCCC12]

3. The agent-method according to claim 1, wherein the organ is kidney or liver.

4. The method according to claim 1, wherein the mammal is a mammal suffering from renal failure or diabetes.

5. The method according to claim 1, wherein the compound is used as a pharmaceutical, a pet food, or a food/drink product.

6. The method according to claim 2, wherein the organ kidney or liver.

7. The method according to claim 2, wherein the mammal is a mammal suffering from renal failure or diabetes.

8. The method according to claim 3, wherein the mammal is a mammal suffering from renal failure or diabetes.

9. The method according to claim 6, wherein the mammal is a mammal suffering from renal failure or diabetes.

10. The method according to claim 2, wherein the compound is used as a pharmaceutical, a pet food, or a food/drink product.

11. The method according to claim 3, wherein the compound is used as a pharmaceutical, a pet food, or a food/drink product.

12. The method according to claim 4, wherein the compound is used as a pharmaceutical, a pet food, or a food/drink product.

13. The method according to claim 6, wherein the compound is used as a pharmaceutical, a pet food, or a food/drink product.

14. The method according to claim 7, wherein the compound is used as a pharmaceutical, a pet food, or a food/drink product.

15. The method according to claim 8, wherein the compound is used as a pharmaceutical, a pet food, or a food/drink product.

16. The method according to claim 9, wherein the compound is used as a pharmaceutical, a pet food, or a food/drink product.
